(19) United States
(12) Patent Application Publication
(10) Pub. No.: US 2022/0071750 A1
(43) Pub. Date: Mar. 10, 2022

(54) ADJUSTABLE MAMMAL LEG BRACE ASSEMBLY

(71) Applicants: TOPMED - CENTRE COLLÉGIAL DE TRANSFERT DE TECHNOLOGIE EN ORTHÈSES, PROTHÈSES ET ÉQUIPEMENTS MÉDICAUX, Québec (CA); UNIVERSITÉ DE MONTRÉAL, Montréal (CA)

(72) Inventors: Marc-André DUSSAULT, Québec (CA); Raphaël BEAUPRÉ-LAFLAMME, Québec (CA); Isabelle TREMBLAY, Québec (CA); Edith MARTIN, Québec (CA); Claude MORIN, Québec (CA); Alvaro GARCIA-BONILLA, Québec (CA)

(21) Appl. No.: 17/416,647

(22) PCT Filed: Dec. 20, 2019
(86) PCT No.: PCT/CA2019/051889

§ 371 Date: Jun. 21, 2021

Related U.S. Application Data
(60) Provisional application No. 62/783,768, filed on Dec. 21, 2018.

(51) Int. Cl. A61D 9/00; A61D 3/00; A61F 5/01

(52) U.S. Cl. CPC A61D 9/00; A61D 2003/006; A61F 5/0102; A61D 3/00

(57) ABSTRACT

The present disclosure concerns an adjustable mammal leg brace comprising: an elongated brace support having a lower portion and a longitudinal direction; a hoof-supporting base engaged with the lower portion of the elongated brace support and extending rearwardly therefrom; and at least one leg-abutting block comprising a leg-receiving surface and being engaged with the elongated brace support above the hoof-supporting base, the at least one leg-abutting block being at least one of pivotably mounted to and translatable along the elongated brace support. It also concerns a brace-receiving stand to receive and support a horse leg brace, comprising: a supporting base having a ground-contacting surface; and a brace-receiving portion pivotably mounted to the supporting base and having a brace-receiving surface. It also concerns a mammal leg brace assembly comprising such an adjustable mammal leg brace and a brace receiving stand, and a kit for forming the same.

### PRIOR APPLICATION

The present application claims priority from U.S. provisional patent application No. 62/783,768, filed on Dec. 21, 2018, and entitled “HORSE LEG BRACE ASSEMBLY”, the disclosure of which being hereby incorporated by reference in its entirety.

### TECHNICAL FIELD

The technical field relates to an animal brace assembly, and more particularly to a mammal leg brace assembly which may be attached to a front leg or to a rear leg of a mammal, for instance of a horse.

### BACKGROUND

A mammal, such as for instance a horse, might get injured or might suffer from diseases impacting its mobility, such as, for instance, arthrosis. A mammal leg brace assembly, configured to support one of the mammal's legs and to maintain it in an adequate position, should then be attached to the leg of the mammal. The mammal leg brace assembly promotes curing of the leg and/or ease the mammal's pain and/or maintain the leg in an adequate position during a medical or surgical act.

However, mammal leg brace assemblies, such as for instance horse leg brace assemblies, are usually dimensioned and designed for specific mammal leg shapes and dimensions, so that they are hardly adaptable to mammals having different leg morphologies.

In view of the above, there is a need for a mammal leg brace assembly which would be able to overcome or at least minimize some of the above-discussed prior art concerns.

### BRIEF SUMMARY

It is therefore an aim of the present invention to address the above-mentioned issues.

According to a general aspect, there is provided an adjustable mammal leg brace comprising an elongated brace support having a lower portion and a longitudinal direction; a hoof-supporting base engaged with the lower portion of the elongated brace support and extending rearwardly therefrom; and at least one leg-abutting block comprising a leg-receiving surface and being engaged with the elongated brace support above the hoof-supporting base, the at least one leg-abutting block being at least one of pivotably mounted to and translatable along the elongated brace support.

According to another general aspect, there is provided an adjustable mammal leg brace comprising an elongated brace support having a longitudinal direction; at least one leg-abutting block comprising a leg-receiving surface and being engaged with the elongated brace support; and a hoof-supporting base engaged with the elongated brace support below the at least one leg-abutting block and extending rearwardly from the elongated brace support, the hoof-supporting base being engageable with the elongated brace support in at least one of a plurality of angular positions and a plurality of longitudinal positions.

According to another general aspect, there is provided a kit for forming an adjustable mammal leg brace according to the present disclosure, wherein the at least one leg-abutting block comprises a leg-abutting pad and a brace support connector engaging the leg-abutting pad with the elongated brace support, wherein the kit further comprises a plurality of interchangeable leg-abutting pads.

According to another general aspect, there is provided a brace-receiving stand to receive and support a mammal leg brace, comprising a supporting base having a ground-contacting surface; and a brace-receiving portion pivotably mounted to the supporting base and having a brace-receiving surface.

According to another general aspect, there is provided an adjustable mammal leg brace assembly comprising an adjustable mammal leg brace comprising: an elongated brace support having a lower portion and a longitudinal direction, a hoof-supporting base engaged with the lower portion of the elongated brace support and extending rearwardly therefrom, and at least one leg-abutting block comprising a leg-receiving surface and being engaged with the elongated brace support above the hoof-supporting base, the at least one leg-abutting block being at least one of pivotably engaged with and translatable along the elongated brace support. The adjustable mammal leg brace assembly further comprises a brace-receiving stand comprising: a supporting base having a ground-contacting surface; and a brace-receiving portion pivotably engaged with the supporting base and having a brace-supporting surface.

According to another general aspect, there is provided an adjustable horse leg brace comprising an elongated support having a lower portion and an upper end, a hoof-supporting base mounted to the lower portion of the elongated support and extending rearwardly therefrom and at least one leg-abutting block at least one of pivotably mounted to and translatable along the elongated support. The at least one leg-abutting block is mounted to the elongated support between the hoof-supporting base and the upper end and the at least one leg-abutting block has a leg-receiving surface.

According to another general aspect, there is provided a brace-receiving stand to receive and support a horse leg brace, comprising a supporting base having a ground contacting surface and a brace-receiving portion pivotably mounted to the supporting base and having a brace-receiving surface.

According to another general aspect, there is provided a horse leg brace assembly comprising an adjustable horse leg brace according to the present disclosure and a brace-receiving stand according to the present disclosure.

According to another general aspect, there is provided a kit for forming an adjustable horse leg brace according to the present disclosure. The kit comprises at least one of a plurality of interchangeable knee-abutting pads, a plurality of interchangeable cannon-abutting pads and a plurality of interchangeable ankle-abutting pads.

### BRIEF DESCRIPTION OF THE DRAWINGS

FIG. 1 is a front perspective view of an adjustable mammal leg brace in accordance with an embodiment, the adjustable mammal leg brace comprising an elongated brace support configured in a first extended configuration;

FIG. 2 is a rear perspective view of the adjustable mammal leg brace of FIG. 1;

FIG. 3 is a front perspective view of a brace-receiving stand in accordance with an embodiment, the brace-receiving stand comprising a telescopic stand assembly configured in a first elongated configuration;

FIG. 4 is a side-elevation view of the brace-receiving stand of FIG. 3, the telescopic stand assembly being configured in a second elongated configuration;

FIG. 5 is a side elevation view of a mammal leg brace assembly comprising the adjustable mammal leg brace of FIG. 1 received and supported by the brace-receiving stand of FIG. 3;

FIGS. 6A to 6C represent a hoof-supporting base and a lower portion of the elongated brace support of the adjustable horse leg brace of FIG. 1, the hoof-supporting base having a supporting base connector configured respectively into first, second and third distinct angular positions with respect to the elongated brace support;

FIG. 7 is a rear perspective view of the adjustable mammal leg brace of FIG. 1, the elongated brace support being configured in a second extended configuration;

FIG. 8 is a rear perspective view, partially exploded, of the adjustable mammal leg brace of FIG. 1;

FIG. 9 is a front top perspective view, partially exploded, of the brace-receiving stand of FIG. 3;

FIG. 10 is a front bottom perspective view, partially exploded, of the brace-receiving stand of FIG. 3;

FIG. 11 is a front perspective view, exploded, of a cannon-abutting block of the adjustable mammal leg brace of FIG. 1;

FIG. 12 is a front perspective view, exploded, of an ankle-abutting block of the adjustable mammal leg brace of FIG. 1; and

FIG. 13 is a front perspective view, sectioned, of the adjustable mammal leg brace of FIG. 1.

### DETAILED DESCRIPTION

In the following description, the same numerical references refer to similar elements. Furthermore, for the sake of simplicity and clarity, namely so as to not unduly burden the figures with several references numbers, not all figures contain references to all the components and features, and references to some components and features may be found in only one figure, and components and features of the present disclosure which are illustrated in other figures can be easily inferred therefrom. The embodiments, geometrical configurations, materials mentioned and/or dimensions shown in the figures are optional and are given for exemplification purposes only.

Moreover, it will be appreciated that positional descriptions such as “above”, “below”, “front”, “rear” “left”, “right” and the like should, unless otherwise indicated, be taken in the context of the figures and correspond to the position and orientation of the mammal leg brace assembly and corresponding parts when being worn by a mammal such as, for instance, a horse. In particular, the terms forward and rearward should be understood with regards to forward and rearward directions of a mammal (for instance with respect to a usual forward walking direction of the mammal). Positional descriptions should not be considered limiting.

To provide a more concise description, some of the quantitative expressions given herein may be qualified with the term “about”. It is understood that whether the term “about” is used explicitly or not, every quantity given herein is meant to refer to an actual given value, and it is also meant to refer to the approximation to such given value that would reasonably be inferred based on the ordinary skill in the art, including approximations due to the experimental and/or measurement conditions for such given value.

In the following description, an embodiment is an example or implementation. The various appearances of “one embodiment”, “an embodiment” or “some embodiments” do not necessarily all refer to the same embodiments. Although various features may be described in the context of a single embodiment, the features may also be provided separately or in any suitable combination. Conversely, although the invention may be described herein in the context of separate embodiments for clarity, it may also be implemented in a single embodiment. Reference in the specification to “some embodiments”, “an embodiment”, “one embodiment” or “other embodiments” means that a particular feature, structure, or characteristic described in connection with the embodiments is included in at least some embodiments, but not necessarily all embodiments.

It is to be understood that the phraseology and terminology employed herein is not to be construed as limiting and are for descriptive purpose only. The principles and uses of the teachings of the present disclosure may be better understood with reference to the accompanying description, figures and examples. It is to be understood that the details set forth herein do not construe a limitation to an application of the disclosure.

Furthermore, it is to be understood that the disclosure can be carried out or practiced in various ways and that the disclosure can be implemented in embodiments other than the ones outlined in the description above. It is to be understood that the terms “including”, “comprising”, and grammatical variants thereof do not preclude the addition of one or more components, features, steps, or integers or groups thereof and that the terms are to be construed as specifying components, features, steps or integers. If the specification or claims refer to “an additional” element, that does not preclude there being more than one of the additional element. It is to be understood that where the claims or specification refer to “a” or “an” element, such reference is not be construed that there is only one of that element. It is to be understood that where the specification states that a component, feature, structure, or characteristic “may”, “might”, “can” or “could” be included, that particular component, feature, structure, or characteristic is not required to be included.

The descriptions, examples, methods and materials presented in the claims and the specification are not to be construed as limiting but rather as illustrative only. Meanings of technical and scientific terms used herein are to be commonly understood as by one of ordinary skill in the art to which the invention belongs, unless otherwise defined. It will be appreciated that the methods described herein may be performed in the described order, or in any suitable order.

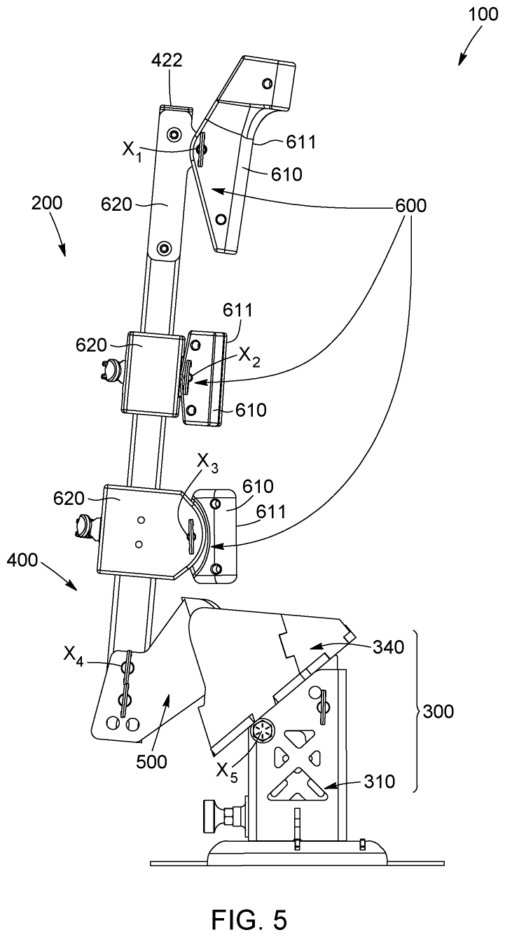

Referring now to the drawings, and more particularly to FIG. 5, there is shown an adjustable mammal leg brace assembly 100 comprising an adjustable mammal leg brace 200 received and supported by a brace-receiving stand 300 and extending upwardly therefrom. The adjustable mammal leg brace 200 is configured to receive and support a leg of a mammal, the leg being either a front leg or a rear leg. In the embodiment shown, the adjustable mammal leg brace assembly 100 is configured to be worn by a horse, so that the following description will usually refer to an adjustable horse leg brace assembly and an adjustable horse leg brace. It should however be understood that a person skilled in the art will have no difficulties to adapt the adjustable horse leg brace of the present disclosure to other animals such as, and without being limitative, cows, calves, and the like.

### Adjustable Mammal Leg Brace

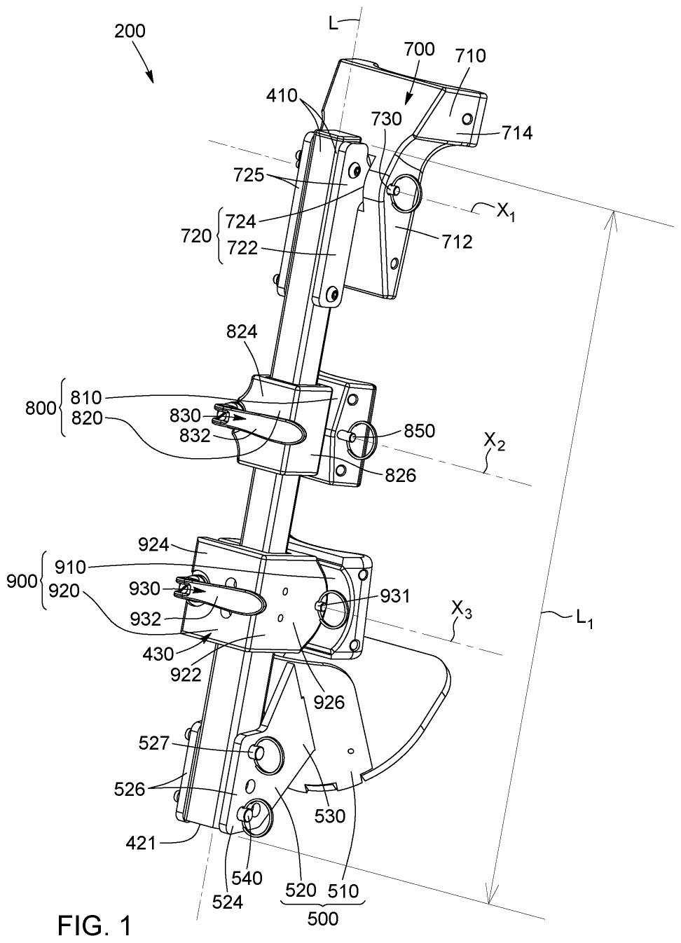

In the embodiment shown, and with reference in particular to FIGS. 1, 2, 7, 8 and 13, the adjustable horse leg brace 200 (or adjustable mammal leg brace) comprises an elongated brace support 400 (or elongated support 400), a hoof-supporting base 500 mounted to (or engaged with, for instance removably engaged with) the elongated brace support 400 and at least one leg-abutting block 600. The at least one leg-abutting block 600 is configured to at least partially abut against a front face of the leg of the horse so that the elongated brace support 400 extends forwardly with respect to the leg of the horse when the adjustable horse leg brace 200 is in use. In the embodiment shown, and without being limitative, the adjustable horse leg brace 200 comprises three leg-abutting blocks 600, namely, and as detailed below, a knee-abutting block 700 (or upper abutting block 700 or upper leg-abutting block 700) configured to abut against an upper portion of the leg of the horse or against a portion of a knee of the horse, a cannon-abutting block 800 (or middle leg-abutting block 800) configured to abut against a portion of a cannon of the horse and an ankle-abutting block 900 (or lower abutting-block 900 or lower leg-abutting block 900) configured to abut against a portion of an ankle of the horse. In the following description, any technical feature relative to one of the knee-abutting block 700, the cannon-abutting block 800 and the ankle-abutting block 900 might also be referred to as the technical feature of one of the leg-abutting blocks 600. It is to be understood that middle, when referring to the cannon-abutting block 800, is not limited to a cannon-abutting block that would be substantially at a center of the portion of the elongated brace support 400 located between the knee-abutting block 700 and the ankle-abutting block 900: the cannon-abutting block 800 might be arranged at any location between the knee-abutting block 700 and the ankle-abutting block 900. As detailed below, and as represented in FIG. 5, each of the leg-abutting blocks 600 comprises a leg-abutting pad 610 defining a leg-receiving surface 611, and a brace support connector 620 mounting (or engaging, or securing, for instance removably) the leg-abutting pad 610 to the elongated brace support 400.

As detailed below and as represented for instance in FIG. 5, each of the leg-abutting blocks 600 is mounted to (i.e. engaged with, for instance removably) the elongated brace support 400 above the hoof-supporting base 500 (considered along a longitudinal direction L of the elongated brace support 400) and, more particularly, is at least one of pivotably mounted to and translatable along the elongated brace support 400.

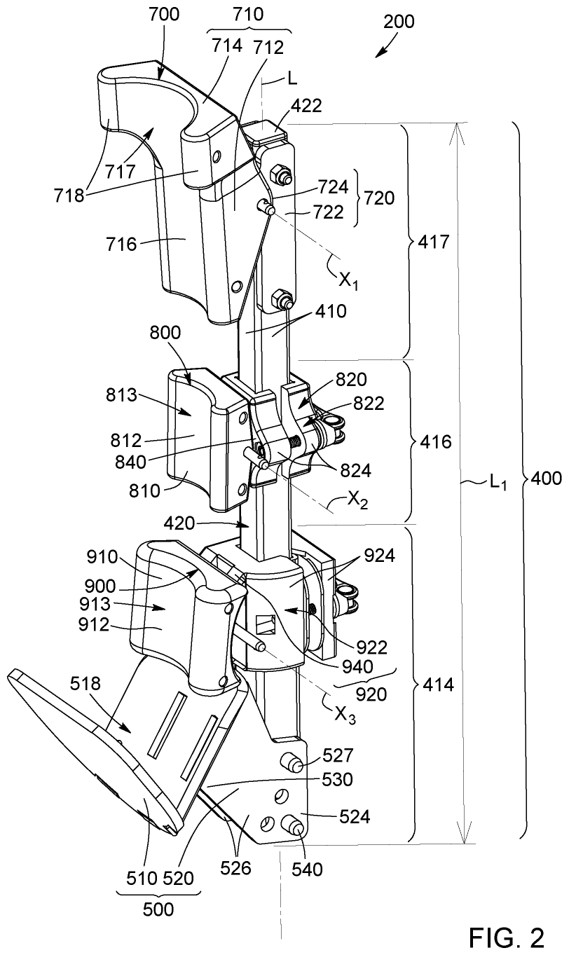

The elongated brace support 400 extends along the longitudinal direction L between a lower end 421 and an upper end 422. The hoof-supporting base 500 is mounted, for instance and without being limitative, to a lower portion 414 of the elongated brace support 400, extending upwardly from the lower end 421 and adjacent thereto. The hoof-supporting base 500 extends rearwardly from the lower portion 414 of the elongated support 400 (with respect to a rearward walking direction of the horse), whereas the leg-abutting block(s) 600 is (are) mounted to the elongated support 400 between the hoof-supporting base 500 and the upper end 422. The above-mentioned leg-receiving surface 611 (FIG. 5) is configured to receive either directly or via a protective layer (such as a bandage—not represented) a portion of the leg of the horse. In the embodiment shown, as represented in FIG. 2, the elongated brace support 400 is dividable, considered along the longitudinal direction L, into an upper portion 417, a central portion 416 and the lower portion 414, wherein the central portion 416 extends between the upper portion 417 and the lower portion 414. In the embodiment shown, there is no materialized boundary between the different portions and each portion does not necessarily extend along a third of a length of the elongated brace support 400.

### Elongated Brace Support

As represented for instance in FIGS. 1, 2 and 8, in the non-limitative embodiment shown, the elongated brace support 400 has a non-circular cross-section (for instance a substantially-squared cross-section in the embodiment shown) and comprises, in the embodiment shown, four peripheral faces 410 extending along the longitudinal direction L, substantially perpendicular to each other. In the embodiment shown, the elongated brace support 400 has a substantially hollow construction, but different constructions could be conceived.

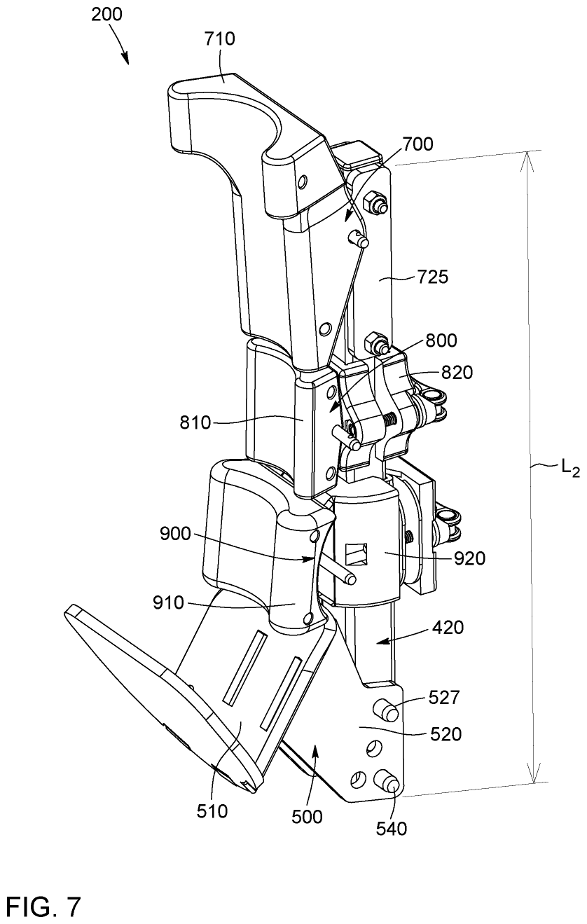

In the embodiment shown, the elongated brace support 400 comprises a telescopic rod assembly 420 comprising for instance an upper brace support segment 425 (or upper segment) and a lower brace support segment 427 (or lower segment) slidably engaged together. In the embodiment shown, the upper and lower segments 425, 427 both have a substantially squared cross-section, so that the upper and lower segments 425, 427 are prevented from pivoting with regards to each other about the longitudinal direction L. The shape and the construction of the upper and lower brace support segments 425, 427 as well as the number of the brace support segments of the telescopic rod assembly 420 are not limited to the embodiment shown. A length of the elongated support 400, considered along the longitudinal direction L, can thus easily be adjusted, for instance to adapt the dimensions of the elongated brace support 400, and thus to adapt the dimensions of the adjustable horse leg brace 200, to the morphology of the horse's leg to be equipped therewith. The elongated brace support 400 is thus configurable into a plurality of extended configurations. The elongated brace support 400 is configurable into at least a first extended configuration, as represented in FIG. 1, wherein the elongated brace support has a first length L1, and into a second extended configuration (or compacted configuration), as represented in FIG. 7, wherein the elongated brace support 400 has a second length L2, smaller than the first length L1. In some embodiments, the second length L2 is smaller than about 90% of the first length L1. In some other embodiments, the second length L2 is smaller than about 80% of the first length L1. In some other embodiments, the second length L2 is smaller than about 70% of the first length L1. In some other embodiments, the second length L2 is smaller than about 60% of the first length L1. In yet some other embodiments, the second length L2 is smaller than about 50% of the first length L1. It is understood that the elongated brace support 400 might be configurable into a plurality of extended configurations, besides the first and second extended configurations represented in the different figures.

The elongated brace support 400 further comprises a lock 430 (or telescopic rod lock 430) for selectively securing the telescopic rod assembly 420 in a selected one of the possible extended configurations. In the embodiment shown, and as described below, the telescopic rod lock 430 is formed by one of the leg-abutting blocks 600.

It is appreciated that the shape, the construction and the configuration of the elongated brace support 400 can vary from the embodiment shown

Leg-Abutting Block

In the embodiment shown, as mentioned above, the adjustable horse leg brace 200 comprises a plurality of leg-abutting blocks 600. The knee-abutting block 700 is mounted proximate the upper end 422 of the elongated brace support 400 whereas the ankle-abutting block 900 is mounted proximate the lower portion 414, for instance between the hoof-supporting base 500 and the knee-abutting block 700. The cannon-abutting block 800 is mounted to the elongated support 400 between the knee-abutting block 700 and the ankle-abutting block 900.

Knee-Abutting Block

In the embodiment shown as represented in FIGS. 1 and 2, the knee-abutting block 700 comprises a knee-abutting pad 710 (or upper leg-abutting pad 710) and a knee-abutting pad connector 720 (or upper brace support connector 720) mounting (for instance pivotably) the knee-abutting pad 710 to the elongated brace support 400, for instance and without being limitative adjacent to the upper end 422 thereof.

In the embodiment shown, the knee-abutting pad 710 comprises a lower portion 712 and an upper portion 714 defining together a knee-receiving surface 716 (or upper leg-receiving surface 716) forming a knee-receiving concavity 717 (or upper leg-receiving concavity 717). In the embodiment shown, the upper portion 714 comprises distal abutting ends 718 protruding rearwardly from the upper brace support connector 720 and rearwardly from the upper leg-receiving surface 716 and dimensioned to partially surround a portion of the knee of the horse, when the adjustable horse leg brace 200 is worn by the horse. In the present disclosure, unless otherwise stated, the term “distal” should be understood with regards to the elongated brace support 400, i.e. extending or located away from the elongated brace support 400. It is appreciated that the shape, the configuration, and the dimensions of the knee-abutting pad 710, as well as the number, the shape and the configuration of the portions forming together the knee-abutting pad 710 can vary from the embodiment shown.

In the embodiment shown, the knee-abutting pad connector 720 (or upper brace support connector 720) comprises a support-engaging portion 722 engageable with the elongated brace support 400, for instance adjacent to the upper end 422 thereof. For instance, the support-engaging portion 722 comprises two substantially parallel support-engaging plates 725 secured (for instance with screws, rivets or any other suitable mechanical fasteners) to two opposed peripheral faces 410 of the elongated support 400.

The knee-abutting pad connector 720 further comprises a knee pad-mounting portion 724 (or upper leg pad-mounting portion 724), for instance protruding rearwardly from the support-engaging portion 722 at an upper portion thereof (with respect to a rearward walking direction of the horse). The knee pad-mounting portion 724 protrudes from the support-engaging portion 722 towards the leg of the horse, when in use. The knee-abutting pad 710 is pivotally mounted to the knee pad-mounting portion 724 about a first pivot axis X1, as represented in FIG. 2. In the embodiment shown, the first pivot axis X1 is substantially perpendicular to the longitudinal direction L of the elongated brace support 400 and is substantially perpendicular to at least one of the peripheral faces 410 of the elongated support 400. In the embodiment shown, the first pivot axis X1 is substantially horizontal when the adjustable horse leg brace 200 is in use.

In the embodiment shown, the knee-abutting pad 710 is removably mounted to the knee-abutting pad connector 720, for instance via a pin 730, or any other suitable removable mechanical fastener. It is thus possible to easily remove the knee-abutting pad 710, for instance to replace it with another knee-abutting pad having different dimensions and/or properties, for it to be better adapted, for instance, to the morphology of the horse equipped with the adjustable horse leg brace 200.

The knee-abutting block 700 might further comprise a securing strap (not represented) removably mountable, for instance, to the knee-abutting pad 710, for surrounding a portion of the leg of the horse when in use, for the knee-abutting block 700 to substantially fit to the knee of the horse. For instance, the securing strap could be engageable with the knee-abutting pad 710 via through openings formed in the knee-abutting pad 710.

It is appreciated that the shape, the construction, the configuration, and the location of the knee-abutting block 700 with regards to the elongated brace support 400, as well as the shape and the configuration of the knee-abutting pad 710 and the knee-abutting pad connector 720 (or upper brace support connector 720) can vary from the embodiment shown. For instance, a knee-abutting block translatable along at least a portion of the elongated support 400 or pivotable about a pivot axis not necessarily substantially perpendicular to the longitudinal direction L of the elongated support 400 could be conceived.

Cannon-Abutting Block

In the embodiment shown, the cannon-abutting block 800 (or middle leg-abutting block 800) comprises a cannon-abutting pad 810 (or middle leg-abutting pad 810) and a cannon-abutting pad connector 820 (or middle brace support connector 820) mounting (for instance pivotably) the cannon-abutting pad 810 to the elongated brace support 400. In the embodiment shown, the cannon-abutting pad connector is further configured to slidably mount the cannon-abutting pad 810 along at least a portion thereof.

In the embodiment shown, the cannon-abutting pad 810 defines a cannon-receiving surface 812 (or middle leg-receiving surface 812), forming a cannon-receiving concavity 813 (or middle leg-receiving concavity 813). It is appreciated that the shape, the configuration, and the dimensions of the cannon-abutting pad 810 can vary from the embodiment shown.

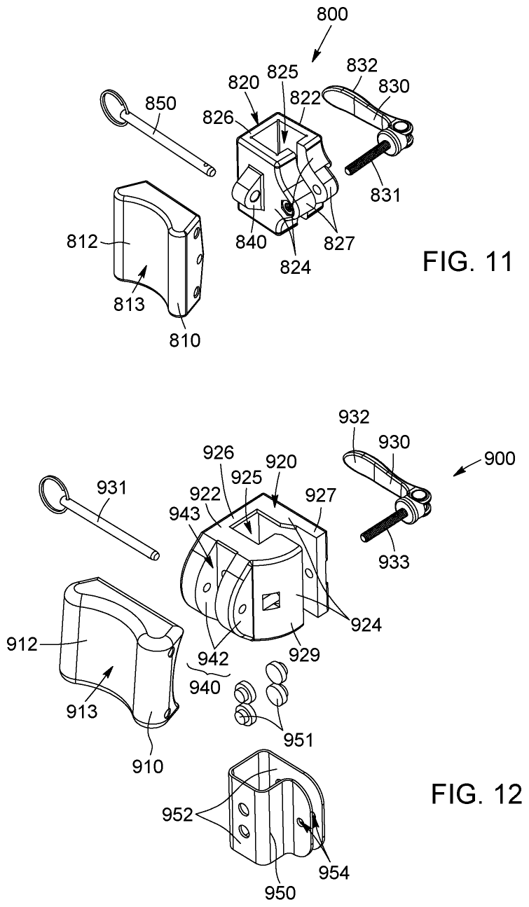

In the embodiment shown, and as represented for instance in FIG. 11, the cannon-abutting pad connector 820 comprises a support-engaging portion 822 engageable with the elongated brace support 400 (for instance removably engageable therewith). For instance, the support-engaging portion 822 has a substantially C-shaped cross-section and comprises two substantially parallel support-engaging jaws 824 facing two opposed peripheral faces 410 of the elongated brace support 400, and a junction portion 826 (or jaw-joining portion 826) facing a third peripheral face 410 of the elongated brace support 400, and connecting together the two support-engaging jaws 824. The support-engaging jaws 824 and the jaw-joining portion 826 define together a brace-support receiving cavity 825 in which the elongated brace support 400 is insertable, so that the support-engaging portion 822 of the cannon-abutting pad connector 820 is engageable with at least a portion of the elongated support 400. It is understood that, in the present description, the expression “C-shaped” is not limited to a support-engaging portion that would define a brace-support receiving cavity having a specific cross-section but refers to any support-engaging portion having a non-continuous shape (i.e. comprising a support-engaging slot or support-receiving opening to allow the engagement of the support-engaging portion with the elongated brace support). It could also be conceived a support-engaging portion that would comprise two or more separate support-engaging elements securable to each other by two or more joining portions.

In the embodiment shown, the support-engaging portion 822 of the cannon-abutting pad connector 820 is slidable along least a portion of the elongated support 400. The brace-support receiving cavity 825 has thus an inner cross-section mating substantially an outer cross-section of the portion of the elongated brace-support 400 to which the cannon-abutting pad connector 820 is mountable.

The cannon-abutting pad 800 further comprises a support connector lock 830, comprising for instance a support connector locking handle 832 or any other suitable mechanical fastener, to lock the support-engaging portion 822 in a selected longitudinal position along the elongated brace support 400.

In the embodiment shown, the support connector lock 830 is configured to move free ends 827 (i.e. ends of the support-engaging jaws 824 opposed to the jaw-joining portion) closer to each other, so as to snugly fit the middle brace support connector 820 around the elongated brace support 400. In the embodiment shown, the support connector lock 830 comprises a locking body 831 (for instance a threaded locking body) engageable into through openings formed in the free ends 827 of the support-engaging jaws 824. The locking handle 832 is thus configured to ease the engagement and displacement of the threaded locking body into the through openings (and to ease the disengagement of the threaded locking body therefrom). In the embodiment shown, the free ends 827 of the jaw-joining portions 824 have substantially similar shapes and dimensions.

The cannon-abutting pad connector 820 (or middle brace support connector 820) further comprises a cannon pad-mounting portion 840 (or middle leg pad-mounting portion 840), for instance protruding rearwardly from the support-engaging portion 822. In the embodiment shown, the cannon-abutting pad 810 is pivotally mounted to the cannon pad-mounting portion 840 about a second pivot axis X2, as represented in FIG. 2. In the embodiment shown, the second pivot axis X2 is substantially perpendicular to the longitudinal direction L of the elongated support 400 and is substantially parallel to the first pivot axis X1. In the embodiment shown, the second pivot axis X2 is substantially horizontal when the adjustable horse leg brace 200 is in use.

In the embodiment shown, the cannon-abutting pad 810 is removably mounted to the cannon-abutting pad connector 820, for instance via a pin 850 insertable into a though opening formed in the middle leg pad-mounting portion 840, or any other suitable removable mechanical fastener. It is thus possible to easily remove the cannon-abutting pad 810, for instance to replace it with another cannon-abutting pad having different dimensions and/or properties, for it to be better adapted, for instance, to the morphology of the horse equipped with the adjustable horse leg brace 200.

It is appreciated that the shape, the configuration, the construction and the location of the cannon-abutting block 800 (or middle leg-abutting block 800) with regards to the elongated brace support 400, as well as the shape and the configuration of the cannon-abutting pad 810 and the middle brace support connector 820 can vary from the embodiment shown. For instance, a cannon-abutting block pivotable about a pivot axis substantially parallel to the longitudinal direction L of the elongated brace support 400 could be conceived.

Ankle-Abutting Block

In the embodiment shown, the ankle-abutting block 900 (or lower leg-abutting block 900) comprises an ankle-abutting pad 910 (or lower leg-abutting pad 910) and an ankle-abutting pad connector 920 (or lower brace support connector 920) mounting the ankle-abutting pad 910 to the elongated brace support 400, for instance and without being limitative to the central portion 416 thereof. In the embodiment shown, the lower brace support connector 920 is configured to slidably mount the ankle-abutting pad 910 along at least a portion of the elongated brace support 400 (for instance along at least the central portion 416 thereof).

As represented for instance in FIG. 12, in the embodiment shown, the ankle-abutting pad 910 defines an ankle-receiving surface 912 (or lower leg-receiving surface 912) forming an ankle-receiving concavity 913 (or lower leg-receiving concavity 913).

In the embodiment shown, the ankle-abutting pad connector 920 (or lower brace support connector 920) is substantially identical to the ankle-abutting pad connector 820. It comprises a support-engaging portion 922 engageable with the elongated support 400 (for instance removably engageable therewith) having a substantially C-shaped cross-section and including two substantially parallel support-engaging jaws 924 facing two opposed peripheral faces 410 of the elongated support 400, and a junction portion 926 (or jaw-joining portion 926) extending therebetween. The elongated support 400 is slidably inserted into a brace support-receiving cavity 925 at least partially defined by the support-engaging jaws 924 and the jaw-joining portion 926.

In the embodiment shown, the support-engaging portion 922 of the lower brace support connector 920 is slidable along least a portion of the elongated brace support 400. The brace-support receiving cavity 925 has thus an inner cross-section mating substantially an outer cross-section of the portion of the elongated brace-support 400 to which the ankle-abutting pad connector 920 is mountable.

The ankle-abutting pad 900 further comprises a support connector lock 930 with a locking handle 932 or any other suitable mechanical fastener, to lock the support-engaging portion 922 in a selected one of the possible longitudinal positions along the elongated support 400.

In the embodiment shown, the support connector lock 930 of the ankle-abutting block 900 is configured to move free ends 927, 929 (i.e. ends of the support-engaging jaws opposed to the jaw-joining portion) closer to each other, so as to snugly fit the lower brace support connector 920 around the elongated brace support 400. In the embodiment shown, the support connector lock 930 comprises a locking body 933 (for instance a threaded locking body) engageable into through openings formed in the free ends 927 of the support-engaging jaws. The locking handle 932 is thus configured to ease the engagement and displacement of the threaded locking body into the through openings (and to ease the disengagement of the threaded locking body therefrom). In the embodiment shown, the free ends 927, 929 of the jaw-joining portions 924 have substantially different dimensions: in the embodiment shown, the pad-facing free end 929 has greater dimensions (namely has a greater width, considered in a plane substantially perpendicular to the longitudinal direction L of the elongated brace support 400) than the forward free-end 927 (considered with respect to a forward walking or running direction of the horse).

In the embodiment shown, the lower leg-abutting pad 900 further comprises a support-engaging sleeve 950 at least partially insertable into the support-receiving cavity 925 and shaped and dimensioned to at least partially surround the portion of the elongated brace support 400 to which the lower brace support connector 920 is engaged. In the embodiment shown, the support-engaging sleeve 950 is securable to an inner surface of the support-engaging portion 922, for instance via pins 951 engageable into through openings formed into wall portions 952 of the support-engaging sleeve 950 and into openings formed in the inner surface of the support-engaging portion 922. The support-engaging sleeve 950 further comprises locking openings 954 in register with the through openings formed in the free ends 927, 929 of the support-engaging jaws 924 of the support-engaging portion 922, when engaged together. The support-engaging sleeve 950 is thus configured to increase the mating of the support-engaging portion 922 with the elongated brace support 400.

Moreover, in the embodiment in which the elongated brace support 400 comprises a telescopic rod assembly 420, the support connector lock 930 can be further configured to secure the telescopic rod assembly 420 in a selected one of the possible extended configurations. In the embodiment in which the telescopic rod assembly 420 comprises upper and lower brace support segments 425, 427 slidably engaged together, the support-engaging portion 922 of the ankle-abutting block 900 can be fixedly engaged with (or fixedly secured to) one of the upper and lower segments 425, 427. The adjustment of the longitudinal position of the ankle-abutting block 900 along the elongated support 400 is thus allowed by the sliding of the upper and lower segments with regards to each other.

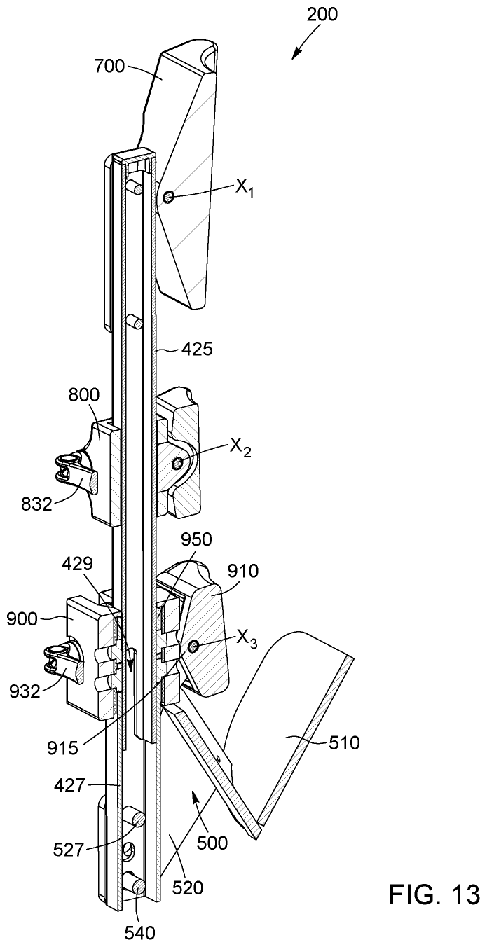

The ankle-abutting pad connector 920 further comprises an ankle pad mounting-portion 940 (or lower leg pad-mounting portion 940), for instance protruding rearwardly from the support-engaging portion 922. In the embodiment show, the ankle pad-mounting portion 940 comprises two substantially parallel ankle pad-mounting plates 942 with facing ankle-pad mounting through apertures formed therein. The ankle pad-mounting plates 942 define therebetween an ankle pad-receiving slot 943 configured to receive a connector-mounting portion 915 of the lower leg-abutting pad 910 (FIG. 13).

In the embodiment shown, the ankle pad-mounting plates 942 and the ankle-abutting pad 910 can be shaped and dimensioned for the ankle-abutting pad 910 to be pivotably mounted to the ankle pad-mounting portion 940 of the lower brace support connector 920 about a third pivot axis X3, for instance substantially parallel to the first and second pivot axes X1, X2. In the embodiment shown, the third pivot axis X3 is substantially horizontal when the adjustable horse leg brace 200 is in use. It could also be conceived an ankle pad-mounting portion and an ankle-abutting pad that would be shaped and dimensioned for the ankle-abutting pad not to pivot with respect to the ankle pad-mounting portion.

In the embodiment shown, the ankle-abutting pad 910 (or lower leg-abutting pad 910) is removably mounted to the ankle-abutting pad connector 920 (or lower brace support connector 920), for instance via a pin 931, or any other removable mechanical fastener. It is thus possible to easily remove the ankle-abutting pad 910, for instance to replace it with another ankle-abutting pad having different dimensions and/or properties, for it to be better adapted, for instance, to the morphology of the horse equipped with the adjustable horse leg brace 200.

It is appreciated that the shape, the configuration, the construction and the location of the ankle-abutting block 900 (or lower leg-abutting block 900) with regards to the elongated brace support 400, as well as the shape and the configuration of the ankle-abutting pad 910 and the ankle-abutting pad connector 920 (or lower brace support connector 920) can vary from the embodiment shown.

Construction

In the embodiment shown, at least one of the leg-abutting pads 610 (namely at least one of the knee-abutting pad 710, the cannon-abutting pad 810 and the ankle-abutting pad 910) is at least partially made of elastomer material, polymer material, such as and without being limitative copolymer material, acetate, polypropylene, high-density polyethylene, polyurethane, nylon material possibly comprising 3D-printed carbon fibers or any other suitable material configured to properly sustain at least a portion of the leg of the horse, without hurting it.

It is thus understood that each of the knee-receiving surface 716, the cannon-receiving surface 812 and the ankle-receiving surface 912 at least partially forms the leg-receiving surface 611 of the leg-abutting blocks 600.

It is further understood that, in the embodiment shown and as described above, at least one of the leg-abutting blocks 600 is removably engaged with the elongated brace support 400.

Hoof-Supporting Base

As represented for instance in FIGS. 1, 2 and 6A to 6C, the hoof-supporting base 500 comprises a hoof-supporting portion 510 configured to support at least a portion of a hoof of the horse, and a base connector 520 (or brace support-connecting portion 520) engageable with (mountable to or securable to, for instance removably) the elongated brace support 400, for instance at the lower portion 414 thereof, below the above-described ankle-abutting block 900.

In the embodiment shown, the hoof-supporting portion 510 comprises a hoof-receiving plate 512 (or hoof-supporting plate 512) substantially planar and a hoof-surrounding peripheral wall 514 having a lower edge 516 secured to the hoof-receiving plate 512 and protruding upwardly therefrom so as to define therewith a hoof-receiving portion 518 (or hoof-receiving cavity 518).

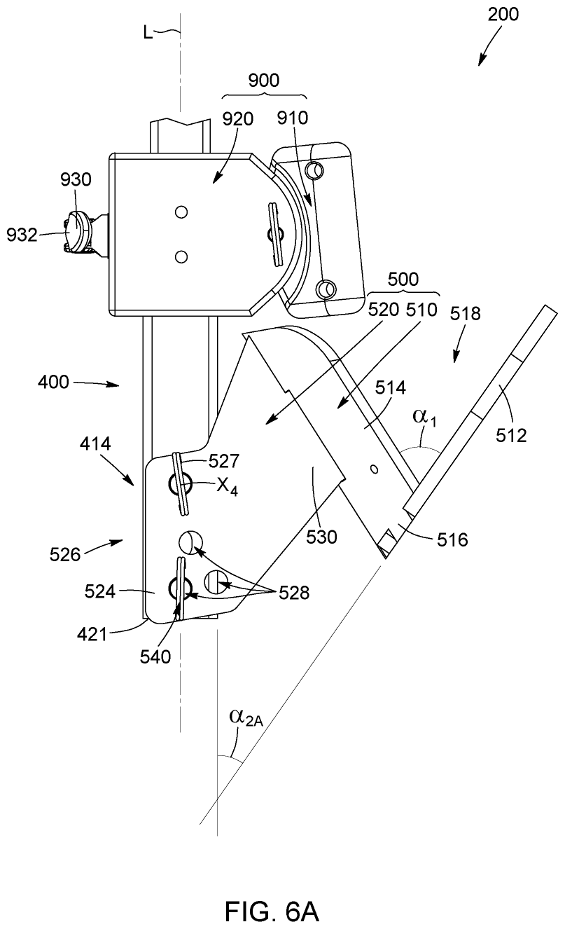

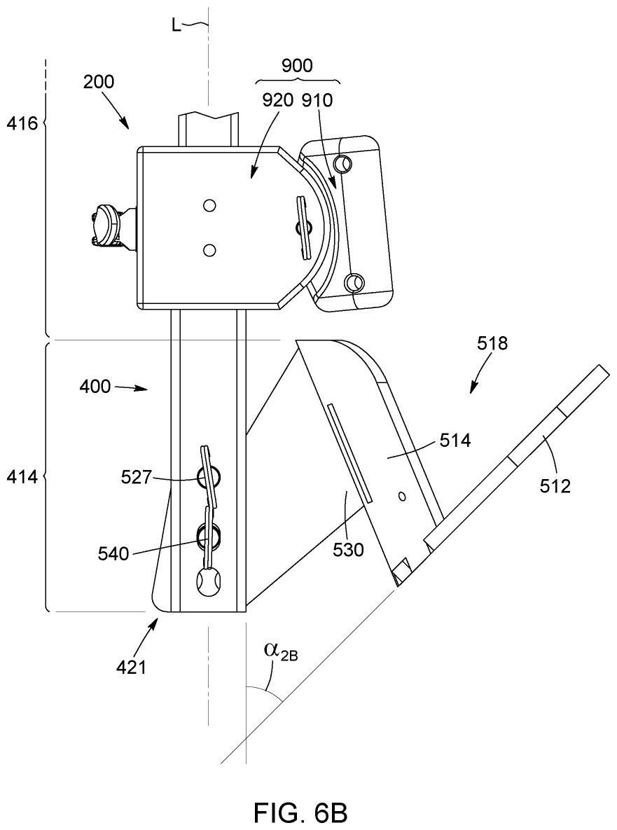

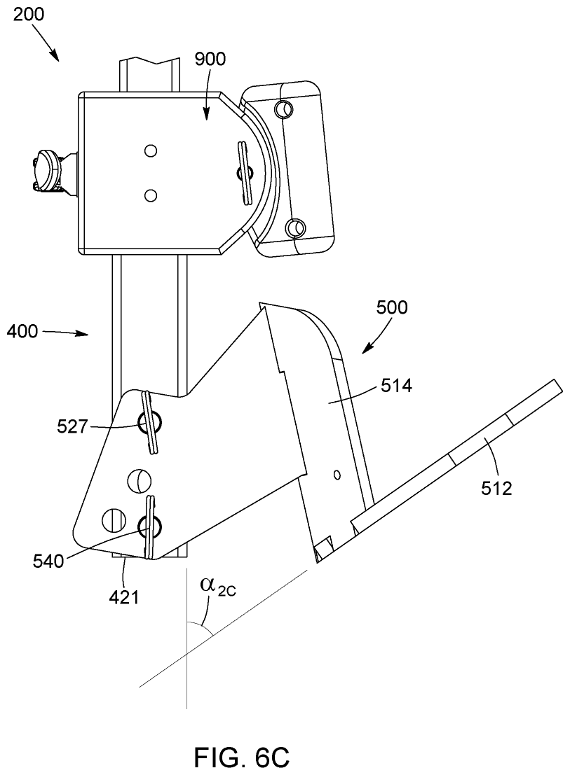

In the embodiment shown, the hoof-surrounding peripheral wall 514 is substantially arcuate so as to define a forward hoof-surrounding concavity (considered with respect to the forward walking direction of the horse) towards the elongated brace support 400 when the hoof-supporting base 500 is mounted thereto. Moreover, as represented in FIG. 6A, the hoof-surrounding peripheral wall 514 forms a first angle α1 (or base angle α1) with the hoof-receiving plate 512. In the embodiment shown, the base angle α1 is acute and is for instance comprised between about 10° and about 80°. In another embodiment, the base angle α1 is comprised between about 30° and about 60°. In another embodiment, the base angle α1 is comprised between about 40° and about 50°.

In the embodiment shown, the base connector 520 (or brace support-connecting portion 520) comprises two substantially parallel support-engaging plates 526 having each a proximal portion 524 (with regards to the elongated brace support 400 when the hoof-supporting base 500 is mounted thereto) (or support-engaging portion 524) and a distal portion 530 (or hoof support-mounting portion 530), the distal portion extending rearwardly from the proximal portion. The proximal portions—or support-engaging portions 524—of the support-engaging plates 526 are secured (or engaged, for instance with screws, rivets or any other suitable mechanical fasteners) to two opposed peripheral faces 410 of the elongated brace support 400. The hoof support-mounting portions 530 of the support-engaging plates 526 are secured (or engaged, for instance by welding or any other suitable mechanical fastener) to an outer face (with regards to the hoof-receiving portion 518) of the hoof-surrounding peripheral wall 514.

As represented in FIGS. 6A to 6C, the brace support-connecting portion 520 is engageable with the elongated brace support 400 in a plurality of base connection angular positions (for instance and without being limitative, three in the embodiment shown). As represented in FIGS. 6A, 6B and 6C, a base connection inclination angle formed between the hoof-receiving plate 512 of the hoof-supporting portion 510 and the longitudinal direction L of the elongated support 400 can be adjusted via the pivoting of the base connector 520 with regards to the elongated support 400, for the inclination angle to respectively and selectively take first, second and third inclination angles α2A, α2B and α2C. In the embodiment shown, the angle formed between the hoof-receiving plate 512 of the hoof-supporting portion 510 and the longitudinal direction L of the elongated brace support 400 is adjustable between about 10° and about 80°. In some other embodiments, the angle is adjustable between about 20° and about 70°. In some other embodiments, the angle is adjustable between about 30° and about 60°. In yet some other embodiments, the angle is adjustable between about 40° and about 50°.

In the embodiment shown, the base connector 520 (or brace support-connecting portion 520) is engageable with the elongated support 400 in three distinct angular positions in which the inclination angles α2A, α2B and α2C are respectively about 35°, as represented in FIG. 6A, about 45°, as represented in FIG. 6B and about 55°, as represented in FIG. 6C. In the embodiment shown, the two support-engaging plates 526 of the base connector 520 have a similar shape, so that the following description of one of the support-engaging plates 526 will apply to any of them.

To configure the hoof-supporting base 500 in a selected one of the plurality of angular positions, the support-engaging plate 526 is pivoted with respect to the elongated support 400 about a fourth pivot axis X4, for instance via an upper aperture formed in the support-engaging plate 526 cooperating with an aperture formed in register in one of the peripheral faces 410 of the elongated brace support 400; a support-mounting pin 527 or any other suitable mechanical fastener engages together the support-engaging plate 526 and the elongated brace support 400. Other apertures could be formed in the elongated support 400 to cooperate with the support-mounting pin 527, so as to engage the support-engaging plate 526 and the elongated support 400 in a plurality of longitudinal positions. As mentioned above, the longitudinal position of the hoof-supporting base 500 with respect to other components of the adjustable horse leg brace 200 (for instance with respect to any one of the leg-abutting blocks 600) can also be adjusted via the sliding with respect to each other of the different members of the telescopic rod assembly 420 of the elongated brace support 400. In the embodiment show, the fourth pivot axis X4 is substantially parallel to the first, second and third pivot axes X1, X2, X3. In the embodiment shown, the fourth pivot axis X4 is substantially horizontal when the adjustable horse leg brace 200 is in use.

The support-engaging plate 526 further comprises a plurality of lower pin-receiving apertures or openings 528 selectively configurable in a configuration wherein one of the different lower pin-receiving apertures 528 is in register with a corresponding aperture defined in one of the peripheral faces 410 of the elongated brace support 400. The hoof-supporting base 500 further comprises a base locker 540 (or base-locking pin 540 or hoof-supporting base locker 540) (comprising in the embodiment shown a pin or any other suitable mechanical fastener) insertable into the apertures (including the selected one of the lower pin-receiving apertures 528) being in register (or aligned) to lock the support engaging plate 526 into the selected one of the plurality of the angular positions, as represented in FIGS. 6A to 6C.

It is thus understood that, in the embodiment shown, the hoof-supporting base 500 is engageable with the elongated brace support 400 in a plurality of angular and longitudinal positions.

In the embodiment shown, the brace support-connecting portion 520 is removably engageable with the elongated brace support 400 so that the hoof-supporting base 500 might be completely separated from the elongated support 400, for instance to easily replace the hoof-supporting base 500 and/or to ease the transport of the adjustable horse leg brace 200.

It is appreciated that the shape, the configuration, the construction and the location of the brace support-connecting portion 520, as well as the shape and the configuration of the support-engaging portions 524 and the hoof support-mounting portions 530 of the support-engaging plates 526 of the brace support-connecting portion 520 can vary from the embodiment shown. A brace support-connecting portion engageable with the elongated support 400 in more or less than three discrete angular positions (for instance continuously pivotable with regards to the elongated support 400) could also be conceived, so as to adapt the adjustable horse leg brace 200 to a plurality of horse morphologies. Moreover, a brace support-connecting portion slidably engageable with at least a portion of the elongated support 400 could also be conceived.

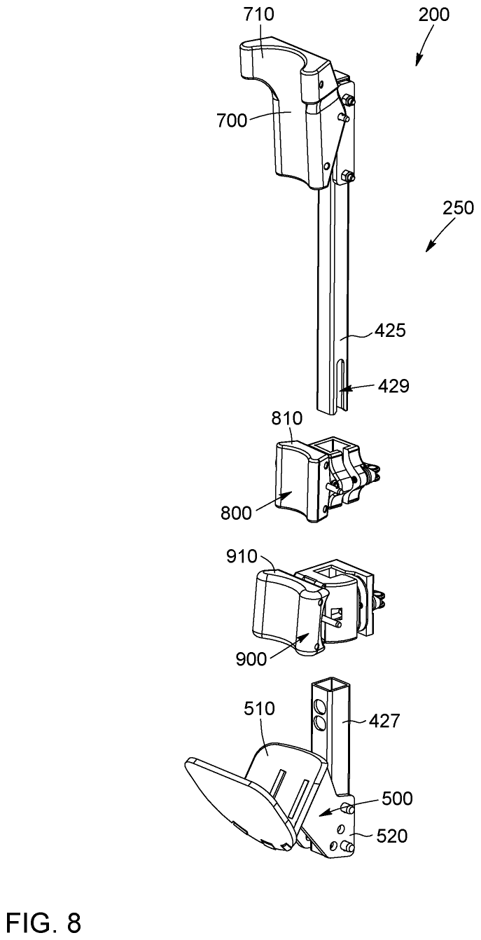

As represented in FIGS. 8 and 13, pin-receiving slots 429 are formed in the upper brace support segment 425 of the telescopic rod assembly 420 to receive the base-locking pin 540 and the support-mounting pin 527 so as not to limit the downward displacement of the upper segment into or around the lower segment 427 when sliding the two segments of the telescopic rod assembly 420 relative to each other.

### Brace-Receiving Stand

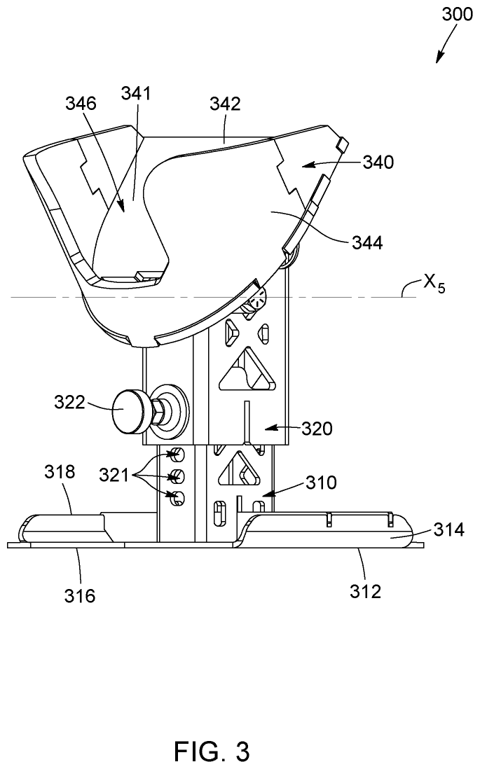

With reference to FIGS. 3, 4, 9 and 10, there is represented a brace-receiving stand 300 comprising a supporting base 310 having a ground-contacting surface 312 and a brace-receiving portion 340 mounted (or engaged, or secured, in a pivotable manner, in the embodiment shown) to the supporting base 310 and having a brace-receiving surface 341. The brace-receiving stand 300 is configured to support the leg of the horse, for instance equipped with a horse leg brace, for instance equipped with the above-described adjustable horse leg brace 200, and to immobilize the leg of the horse, for instance during a medical or surgical act.

Supporting Base

In the embodiment show, the supporting base 310 comprises a ground-contacting plate 314 having a lower surface 316 forming the ground-contacting surface 312, and an opposed upper surface 318. The ground-contacting plate 314 is dimensioned to ensure a stable support of the brace-receiving stand 300 on the ground.

It is appreciated that the shape and the configuration of the supporting base 310 can vary from the embodiment shown.

Brace-Receiving Portion

In the embodiment shown, and as represented in FIGS. 3, 4, 9 and 10, the brace-receiving portion 340 comprises a brace-receiving plate 342 (or brace-supporting plate 342) having an upper surface forming at least partially the brace-receiving surface 341 (or brace-supporting surface 341). The brace-receiving portion 340 further comprises a brace-surrounding peripheral wall 344 extending upwardly from the brace-receiving plate 342 and defining therewith a brace-receiving cavity 346.

It is understood that the brace-receiving cavity 346 is shaped and dimensioned for the hoof-supporting base 500 of the adjustable horse leg brace 200 to be at least partially received therein. In the embodiment shown, as represented in FIG. 5, the brace-receiving cavity of the brace-receiving portion 340 is dimensioned for the hoof-receiving plate of the hoof-supporting portion of the hoof-supporting base 500 to face and to be at least partially supported by the brace-receiving plate of the brace-receiving portion 340 when the hoof-supporting base 500 is at least partially received in the brace-receiving cavity. Moreover, the brace-surrounding peripheral wall portion 344 is shaped and dimensioned for the hoof-surrounding peripheral wall 514 of the hoof-supporting base 510 to be at least partially abutted against the brace-surrounding peripheral wall portion 344 when the hoof-supporting base 500 is received in the brace-receiving cavity 346.

In the embodiment shown, the brace-receiving portion 340 further comprises an upper stand member 323 and a stand connector 350, for instance protruding downwardly from a lower surface of the brace-receiving plate 342 and configured to engage the brace-receiving plate 342 to the upper stand member 323. The stand connector 350 is engageable with the upper stand member 323 in a plurality of angular positions. In other words, the stand connector 350 is configured to cooperate with the upper stand member 323, for instance with an upper portion 311 thereof, to pivotably mount the brace-receiving plate 342 to the upper stand member 323, and thus to pivotably mount the brace-receiving portion 340 to the supporting base 310. In the embodiment shown, the stand connector 350 is pivotably mounted to the upper stand member 323 about a fifth pivot axis X5, for instance extending substantially horizontally. The brace-receiving stand 300 further comprises a stand locker 352 (comprising a stand-locking pin in the embodiment shown) selectively cooperating, for instance, with locker-receiving openings 353 formed in the upper portion 311 of the upper stand member 323 for selectively securing the brace-receiving portion 340 in a plurality of angular configurations (two, in the embodiment shown) with respect to the supporting base 310.

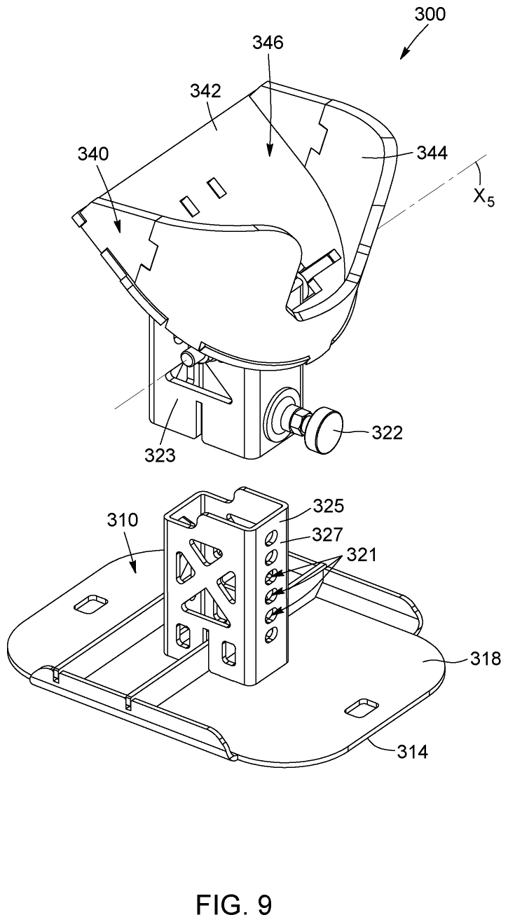

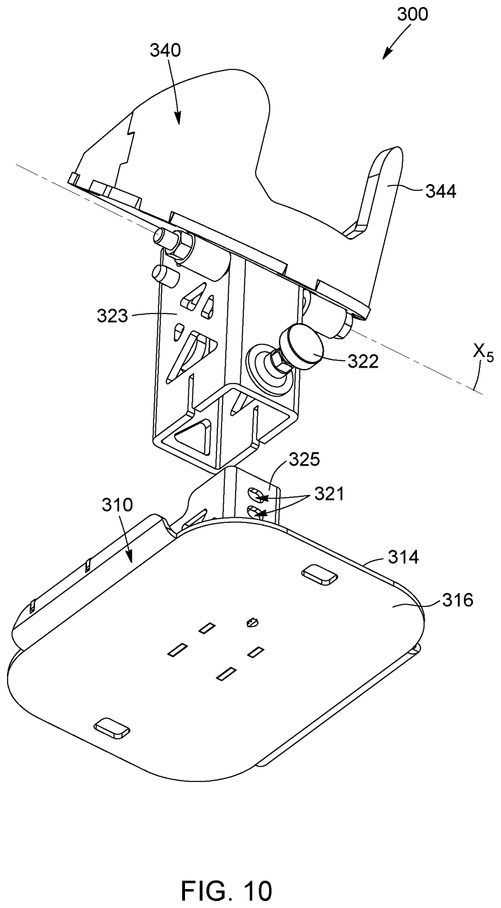

The brace-receiving stand 300 further comprises a telescopic stand assembly 320 extending upwardly (for instance substantially perpendicularly), in the embodiment shown, from the upper surface 318 of the supporting plate 314. As represented in FIGS. 9 and 10, the telescopic stand assembly 320 comprises the above-mentioned upper stand member 323 and a lower stand member 325 protruding upwardly (for instance substantially perpendicularly) from the upper surface of the ground-contacting plate 314. The upper and lower stand members 323, 325 are slidably engaged together for the telescopic stand assembly 320 to be configurable into a plurality of elongated configurations. In the embodiment shown, the lower stand member 325 is slidably engageable into the upper stand member 323.

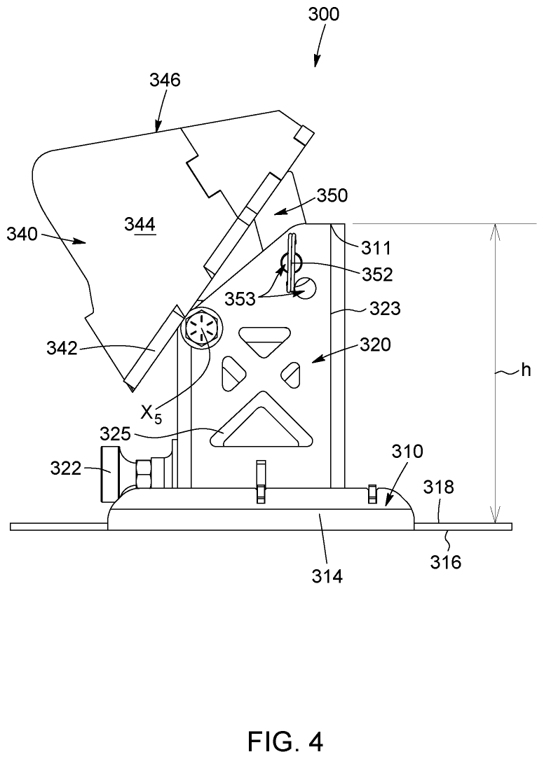

The brace-receiving stand 300 further comprises a telescopic stand locker 322 for selectively securing the telescopic stand assembly 320 in a selected one of the plurality of elongated configurations, so as to adjust a height h of the brace-receiving stand 300 (the height h being defined, as represented in FIG. 4, as the distance between the upper end 311 of the upper stand member 323 and the ground-supporting plate 314). In the embodiment shown, the telescopic stand locker 322 is selectively engageable into each one of a plurality of locker-receiving openings 321 formed in a wall portion 327 of the lower stand member 325 of the telescopic stand assembly 320.

It is appreciated that the shape and the configuration of the brace-receiving stand 300, as well as the shape, the configuration, and the location of the brace-receiving portion 340 with regards to the supporting base 310 as well as the shape, the configuration and the number of stand members of the telescopic stand assembly can vary from the embodiment shown.

It is thus understood that the horse leg brace assembly 100 according to the present disclosure is configured to be adapted to many horse morphologies. To this end, as described above, the adjustable horse leg brace assembly 100 allows adjusting the length of the elongated brace support 400, adjusting the angular position and/or the longitudinal position of at least one of the leg-abutting blocks 600 and/or the hoof-supporting base 500, adjusting the height h of the brace-receiving stand 300 and/or adjusting the inclination of the brace-receiving portion 340 with regards to the supporting base 310.

### Kit

According to another aspect of the disclosure, as represented in FIG. 8, there is provided a kit 250 for forming an adjustable horse leg brace 200 according to the present disclosure. The kit 250 might further comprise a plurality of interchangeable leg-abutting pads 610, such as at least one of a plurality of interchangeable knee-abutting pads 710, a plurality of interchangeable cannon-abutting pads 810 and a plurality of interchangeable ankle-abutting pads 910. At least some of the interchangeable leg-abutting pads 610 might have different widths, different resistance, elasticity and/or softness properties and/or be made of different materials. It is thus understood that the use of interchangeable leg-abutting pads 610 further improve the adaptability of the adjustable horse leg brace 200 to a plurality of horse morphologies.

The kit 250 might further be configured to form an adjustable horse leg brace assembly 100 according to the present disclosure and might thus further comprise a brace-receiving portion 340 and a supporting base 310 so as to form a brace-receiving stand 300.

Several alternative embodiments and examples have been described and illustrated herein. The embodiments of the invention described above are intended to be exemplary only. A person of ordinary skill in the art would appreciate the features of the individual embodiments, and the possible combinations and variations of the components. A person of ordinary skill in the art would further appreciate that any of the embodiments could be provided in any combination with the other embodiments disclosed herein. It is understood that the invention may be embodied in other specific forms without departing from the central characteristics thereof. The present examples and embodiments, therefore, are to be considered in all respects as illustrative and not restrictive, and the invention is not to be limited to the details given herein. Accordingly, while the specific embodiments have been illustrated and described, numerous modifications come to mind. The scope of the invention is therefore intended to be limited by the scope of the appended claims.

What is claimed is:

1.-72. (canceled)

73. An adjustable mammal leg brace comprising:
an elongated brace support having a lower portion and a longitudinal direction;
a hoof-supporting base engaged with the lower portion of the elongated brace support and extending rearwardly therefrom; and
at least one leg-abutting block comprising a leg-receiving surface and being engaged with the elongated brace support above the hoof-supporting base, the at least one leg-abutting block being at least one of pivotably mounted to and translatable along the elongated brace support.

74. The adjustable mammal leg brace according to claim 73, wherein at least one of the hoof-supporting base and the at least one leg-abutting block is removably engaged with the elongated brace support.

75. The adjustable mammal leg brace according to claim 73, wherein the elongated brace support comprises a telescopic rod assembly comprising upper and lower brace support segments slidably engaged together for the telescopic rod assembly to be configurable into a plurality of extended configurations and wherein the elongated brace support further comprises a telescopic rod lock engageable with the telescopic rod assembly to configure the telescopic rod assembly in a selected one of said plurality of extended configurations.

76. The adjustable mammal leg brace according to claim 73, wherein the at least one leg-abutting block comprises:
a leg-abutting pad defining the leg-receiving surface, the leg-receiving surface defining a leg-receiving concavity; and
a brace support connector connecting the leg-abutting pad to the elongated brace support.

77. The adjustable mammal leg brace according to claim 76, wherein the brace support connector comprises a support-engaging portion slidably mounted to the elongated brace support for the brace support connector to be configurable into a plurality of longitudinal positions along the elongated brace support and a leg pad-mounting portion extending rearwardly from the support-engaging portion.

78. The adjustable mammal leg brace according to claim 77, wherein the leg-abutting pad is at least one of removably and pivotably mounted to the leg pad-mounting portion and pivotable about a leg pad-pivoting axis substantially perpendicular to the longitudinal direction of the elongated brace support.

79. The adjustable mammal leg brace according to claim 73, wherein the at least one leg-abutting block comprises at least one of a knee-abutting block, a cannon-abutting block and an ankle-abutting block.

80. An adjustable mammal leg brace comprising:
an elongated brace support having a longitudinal direction;
at least one leg-abutting block comprising a leg-receiving surface and being engaged with the elongated brace support; and
a hoof-supporting base engaged with the elongated brace support below the at least one leg-abutting block and extending rearwardly from the elongated brace support, the hoof-supporting base being engageable with the elongated brace support in at least one of a plurality of angular positions and a plurality of longitudinal positions.

81. The adjustable mammal leg brace according to claim 80, wherein the hoof-supporting base comprises a hoof-supporting portion and a brace support-connecting portion removably engageable with the elongated brace support.

82. The adjustable mammal leg brace according to claim 81, wherein the hoof-supporting portion comprises a hoof-supporting plate and a hoof-surrounding peripheral wall secured to the hoof-supporting plate and defining therewith a hoof-receiving cavity.

83. The adjustable mammal leg brace according to claim 82, wherein the hoof-surrounding peripheral wall is substantially arcuate and defines a forward hoof-surrounding concavity.

84. The adjustable mammal leg brace according to claim 82, wherein the hoof-surrounding peripheral wall defines an acute base angle with the hoof-supporting plate.

85. The adjustable mammal leg brace according to claim 81, wherein the brace support-connecting portion is pivotably engaged with the elongated brace support and pivotable about a base pivot axis substantially perpendicular to the longitudinal direction of the elongated brace support.

86. The adjustable mammal leg brace according to claim 81, wherein the brace support-connecting portion is engageable with the elongated brace support in said at least one of a plurality of angular positions and a plurality of longitudinal positions and wherein the hoof-supporting base comprises a hoof-supporting base locker engageable with the brace support-connecting portion to configure the brace support-connecting portion in a selected one of said at least one of a plurality of angular positions and a plurality of longitudinal positions.

87. A brace-receiving stand to receive and support a mammal leg brace, comprising:
a supporting base having a ground-contacting surface; and
a brace-receiving portion pivotably mounted to the supporting base and having a brace-receiving surface.

88. The brace-receiving stand according to claim 87, wherein the supporting base comprises a ground-contacting plate and wherein the brace-receiving stand comprises a telescopic stand assembly extending upwardly from the ground-contacting plate and configurable into a plurality of elongated configurations.

89. The brace-receiving stand according to claim 87, wherein the brace-receiving portion comprises a brace-supporting plate and a brace-surrounding peripheral wall extending upwardly from the brace-supporting plate and defining therewith a brace-receiving cavity, and wherein the brace-receiving portion comprises a stand connector protruding downwardly from the brace-supporting plate and mountable to the supporting base in a plurality of angular positions.

90. An adjustable mammal leg brace assembly comprising:
the adjustable mammal leg brace according to claim 73; and
a brace-receiving stand comprising:
a supporting base having a ground-contacting surface; and
a brace-receiving portion pivotably engaged with the supporting base and having a brace-supporting surface.

91. The adjustable mammal leg brace assembly according to claim 90, wherein the brace-receiving portion comprises a brace-supporting plate and a brace-surrounding peripheral wall extending upwardly from the brace-supporting plate and defining therewith a brace-receiving cavity, wherein the hoof-supporting base of the adjustable mammal leg brace is at least partially received into the brace-receiving cavity, wherein the hoof-supporting base comprises a hoof-supporting portion and a brace support-connecting portion removably engageable with the elongated brace support, and wherein the hoof-supporting portion faces and is at least partially supported by the brace-supporting plate.

92. The adjustable mammal leg brace assembly according to claim 91, wherein the hoof-supporting portion comprises a hoof-supporting plate and a hoof-surrounding peripheral wall extending upwardly from the hoof-supporting plate and defining therewith a hoof-receiving cavity and wherein the hoof-surrounding peripheral wall of the hoof-supporting base is shaped and dimensioned to be at least partially abutted against the brace-surrounding peripheral wall.
